(19) United States
(12) Plant Patent Application Publication
(10) Pub. No.: US 2002/0095704 P1
(43) Pub. Date: Jul. 18, 2002

(54) Chrysanthemum plant named 'Chanoud'

(76) Inventor: Jean Pierre Challet, Nuaille (FR)

Correspondence Address: BURNS DOANE SWECKER & MATHIS L L P, POST OFFICE BOX 1404, ALEXANDRIA, VA, 22313-1404, US

(21) Appl. No.: 09/759,353

(22) Filed: Jan. 16, 2001

(51) Int. Cl. A01H 5/00

(52) U.S. Cl. PLT/289

(57) ABSTRACT

A new and distinct October-flowering Chrysanthemum cultivar is provided. Attractive light yellow double blossoms are formed in profusion. The growth habit is low compact and well-branched and yields a generally uniform and spherical overall plant. The dark green leaves contrast well with the light yellow blossoms. The new cultivar is particularly well suited for growing as an attractive potted gift plant under greenhouse growing conditions.

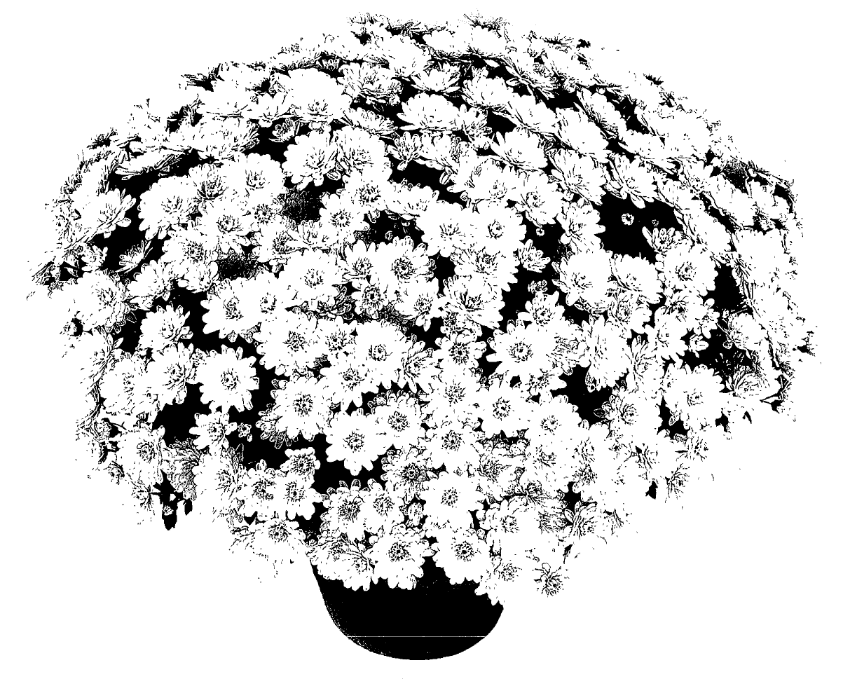

### SUMMARY OF THE INVENTION

The present invention comprises a new and distinct cultivar of Chrysanthemum, botanically known as Dendranthema grandiflora, and hereinafter is referred to by the cultivar name ‘Chanoud’.

The new cultivar of the present invention was created at Nuaille, France during 1994 when plants of the ‘Chadixi’ cultivar (non-patented in the United States) were irradiated with gamma irradiation. Cuttings from the irradiated plant were used to produce new plants that were carefully studied. The new cultivar was selected during 1994 from among the rooted cuttings because of its distinctive combination of characteristics.

It was found that the new Chrysanthemum cultivar of the present invention displays:

(a) a low compact, well-branched, and generally uniform and spherical growth habit,

(b) during October a profusion of attractive decorative light yellow double blossoms,

(c) dark green leaves that contrast well with the light yellow blossoms, and

(d) an ability to grow well in pots to provide an attractive potted gift plant.

The new cultivar is considered to be primarily an October-flowering greenhouse cultivar with natural flowering in weeks 42 to 43 at Nuaille, France. It can be grown well either singly or in clumps in pots. Also, the new cultivar is suited for growing in the landscape where it has withstood temperatures of 0° to −2° C. The blossoms commonly last in excess of three weeks on the plant. Pinching is helpful to further enhance branching; however, such pinching is not necessary since the plant already is naturally self-branching.

The new cultivar can be readily distinguished from its ‘Chadixi’ parent that exhibits purple blossoms.

Asexual reproduction of the new cultivar by cuttings initially taken during 1994 as performed at Nuaille, France, in a controlled environment has demonstrated that the characteristics of the new cultivar as herein described are firmly fixed and are retained through successive generations of asexual progation.

The new cultivar also was tested during 1997 at Cambridge, Great Britain.

‘Chanoud’ has not been observed under all possible environmental conditions to date. Accordingly, it is possible that the phenotype may vary somewhat with variations in the environment, such as temperature, light, day length, contact with pesticides and/or subjection to growth regulation treatments.

### BRIEF DESCRIPTION OF THE PHOTOGRAPH

The accompanying photograph was obtained during October, 1996 and depicts three plants of the new cultivar while growing in a pot in a greenhouse at Nuaille, France. The plants were rooted during June, 1996, and accordingly were approximately four months of age. The generally spherical growth habit and the profusion of attractive light yellow double decorative blossoms with dark green foliage are illustrated.

### DETAILED DESCRIPTION

The chart used in the identification of colors described hereafter is the R.H.S. Colour Chart of The Royal Horticultural Society, London, England. In some instances more common color terms are provided and are to be accorded their usual dictionary significance. The plants described were grown in 20 cm. pots in greenhouses at Nuaille, France, three plants to a pot, and were rooted in mid-June, and were observed on October 20^{th}. No growth regulation was used. The growing conditions approximated those commonly utilized for the commercial production of decorative pot mums.

Classification:

Botanical.—Dendranthema grandiflora, cv. ‘Chanoud’.

Commercial.—Decorative pot mum.

Inflorescence:

A. Capitulum

Type.—Decorative.

Diameter across face.—Approximately 25 to 35 mm on average when fully expanded.

Outside bud coloration.—Yellow Group 10B.

B. Corolla florets

General tonality.—Outer Side: Yellow Group 5D with some very light red that increases slightly towards the base. —Inner Side: Yellow Group 5D. In contrast the floret coloration of the parent ‘Chadixi’ variety is Purple Group 75C with some slightly deeper shading and edges of Purple Group 75B.

Configuration ray florets.—Petaloid.

Disc florets.—None.

C. Reproductive organs

Androecium.—Present with ray florets.

Gynoecium.—Present with ray florets.

Pollen.—Generally formed in only a slight quantity at the end of blooming and yellow-orange in coloration.

Fragrance.—Weak and typical of Chrysanthemum.

Plant:

A. General appearance

Height.—Very short, and approximately 35 cm on average at four months of age.

Width.—Approximately 55 cm on average at four months of age.

B. Foliage

Color.—Upper Surface: Generally between Green Group 137A and Green Group 139A. —Under Surface: Generally between Green Group 137A and Green Group 139A with slightly more grey.

Size.—The leaves at the tip of the stem commonly are approximately 3.5 cm in length and commonly are approximately 1.7 cm in width. The leaves at the base of the stem commonly are approximately 10 cm in length and commonly are approximately 5 cm in width.

Texture.—Fleshy.

Thickness.—Medium.

Serration.—Fine.

Shape of base of leaf.—Obtuse.

Shape of tip of leaf.—Mucronate.

Claw in base of sinus between lateral lobes.—Absent.

Stems.—Thin to medium and generally round in cross-section, strongly rigid, and commonly Yellow-Green Group 146C in coloration with no anthocyanin coloration.

Internode length.—Very short, and commonly approximately 5 to 10 mm.

I claim:

1. A new and distinct cultivar of Chrysanthemum plant that displays:
(a) a low compact, well-branched, and generally uniform and spherical growth habit,
(b) during October a profusion of attractive decorative light yellow double blossoms,
(c) dark green leaves that contrast well with the light yellow blossoms, and
(d) an ability to grow well in pots to provide an attractive potted gift plant;
substantially as herein shown and described.
